(19) United States
(12) Patent Application Publication
(10) Pub. No.: US 2009/0294604 A1
(43) Pub. Date: Dec. 3, 2009

(54) POLE GRIPPING HOOK FOR MEDICAL SUPPLIES

(76) Inventor: MARK SUNDERLAND, Ottawa (CA)

Correspondence Address: BLAKE, CASSELS & GRAYDON, LLP, 45 O'CONNOR ST., 20TH FLOOR, OTTAWA, ON, K1P 1A4, CA

(21) Appl. No.: 12/128,015

(22) Filed: May 28, 2008

(51) Int. Cl. A61M 5/14; F16M 13/02

(52) U.S. Cl. 248/124.1; 248/226.11; 248/227.3

(57) ABSTRACT

A bracket slides on a vertical pole and grips the pole at any position to support a multiplicity of devices at various heights and extending in various directions, such as medical devices and medical fluids. The pole may be on wheels as used in hospitals in the delivery of treatment to patients, such as IV fluids and drugs that may use pumps that are also supported on the pole.

### BACKGROUND OF THE INVENTION

1. Field of the Invention

The present invention relates to the provision of medical treatment to patients, usually in hospitals, involving fluids to be delivered intravenously, pumps for such fluids, and monitors of a patient's condition.

2. Description of Related Art

As technology plays an increasingly important role at the bedside in a hospital and for patients in general, the need to physically support equipment also increases. Such equipment, which will here be collectively called paraphernalia, comprises without limitation pumps, monitors, lamps, intravenous and infusion reservoirs which may be bottles or bags, and reservoirs for draining body fluids. All require physical support in proximity to a patient.

The current practice is to move a portable pole to a position close to the bed. These are often referred to as I.V. poles, because solutions to be injected intravenously are the most common paraphernalia on the pole, but in fact the poles have broader uses. The pole is normally equipped with one or two hooks at the top on which to hang reservoirs of various fluids. These poles are usually telescopic, so that the height can be varied. The height is required to vary to accommodate various devices, or to alter the hydrostatic pressure of a fluid in a gravity feed system of a dispensing reservoir, or to be within easy reach of the operator, and the variation is achieve by telescoping the upper portion of the pole into the tubular lower portion. The position is held by tightening a screw that passes through the lower pole and bears on the part of the pole that is within the pole. A disadvantage of such designs is that it requires two hands to vary the height, and the supporting hooks are only at the top. A further disadvantage is that the supporting hooks are an integral part of the portable pole and limited in number, usually to two hooks at the top.

In another example of current practice, such as disclosed in U.S. Pat. No. 4,702,488, the hooks for supporting bottles or bags of intravenous solutions are mounted on a clamp that attaches to the vertical pole at any height by means of a screw that can be tightened to bear down on the pole. More complicated screwed-on clamps are disclosed in U.S. Pat. Nos. 6,079,678, 5,322,253, and 6,913,234. The common feature throughout the prior art is that clamps, or a telescoping pole, are held in place by the pressure of a screw onto a pole.

In addition to the versatility of supporting equipment at any height or orientation on a pole, this invention addresses the concern for infection control. For this reason, to facilitate cleaning, all the tubing and support modules have smooth surfaces and no crevices. Also, the material of the support modules may have an inherent anti-microbial property.

By eliminating telescopic joints and concealed tubing in the pole, and also eliminating screws threads and thumbwheel screw heads, the present invention offers the benefit of single hand operation and also eliminates large areas, such as screw threads, that can harbour contamination such as pathogens.

### SUMMARY OF THE INVENTION

The present invention makes use of a single vertical pole, which may be a solid rod or a tube, of uniform diameter. The pole is mounted on a supporting base, usually having wheels, or attached to other equipment such as a bed, trolley or gurney. One or more support modules can be added to the pole to support paraphernalia. We use the term “support module” or simply “module” because a pole may be set up with one, two, or a great many modules, and there may be different types of modules, so the system as a whole is modular. Each module contains a hole, called the “pole hole”, through which the pole passes. Each module can be placed at, and will remain placed at, any height or orientation between the top and bottom of the pole.

A significant feature is that the positioning of modules is dependent on friction alone and not on notching in the pole and not on the pressure of a screw onto the pole.

The modules can be freely positioned by one hand to any desired point on the pole and the modules will hold their position by friction alone. The weight of the module itself, and especially the added weight of anything attached at or near the tip of the module, will cause the module to rotate a few degrees in the vertical plane so it is not orthogonal to the pole. The rim of the pole hole then binds against the pole so the support module will not slide down the pole. The grip of the modules to the pole increases as the weight loaded on the module increases. If the module is rotated back to the position orthogonal to the pole, the rim of the pole hole is no longer pressed at an angle against the pole and the module can be slid up or down the pole. Rotating the module to the orthogonal position is most conveniently done by grasping the module at the end near the pole.

The module when unloaded has little weight, so it would not reliably hold its position on the pole by means of friction with the rim of the pole hole. The present invention provides gripping means inserted in the pole hole to press against the pole and so to hold the unloaded module in position.

Since the gripping of the pole by a module is due to the levering effect of the weight of the paraphernalia supported by the module, this invention functions best when there is only one hook on a module, extending in one direction, although it would be possible to have more than one hook projecting from the same module at the end farther from the pole. It is generally convenient to obtain the benefit of a second hook by adding an additional single-hook module on the pole.

### DESCRIPTION OF THE DRAWINGS

FIG. 1 shows the entire assembly of pole and several modules

FIG. 2 shows the top view of the basic module.

FIG. 3 shows a sectional view of the module and the springy insert and the bow spring.

FIG. 4 shows a side view of the springy insert and bow spring.

FIG. 5 shows the underside view of the springy insert.

FIG. 6 shows the underside of the module with the springy insert in position.

FIG. 7 shows a the top view of the module that has the rod hole but not the hook.

FIG. 8 shows a section of the module having a rod for connection to other devices.

FIG. 9 shows the rod that is used for connection to other devices.

FIG. 10 shows an apparatus for adding additional poles so more modules can be used.

FIG. 11 shows details of the construction of the additional poles in an exploded view, in which top and bottom cross-members are shown both as side views and end views

### DETAILED DESCRIPTION OF THE INVENTION

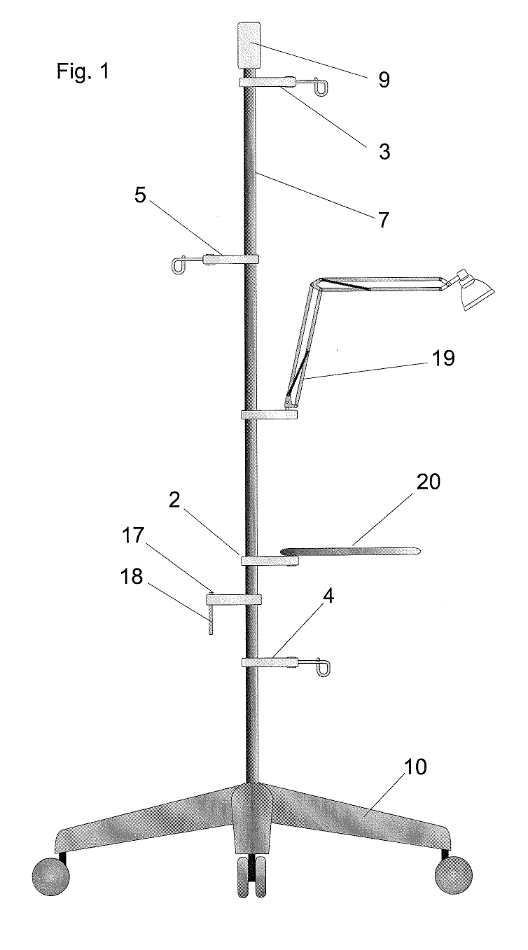

FIG. 1 shows a typically assembly in which a pole 7 is mounted vertically on a wheeled base 10. The pole is surmounted with a cap 9 which serves several purposes. It seals the end of the pole 7, if the pole is a hollow tube, to prevent entry of contamination. It can bear colour or markings that identify the user, or the intended use, or the department of an institution to which the apparatus belongs, or simply an inventory number. The cap 9 is preferably fitted tightly, to prevent easy or inadvertent removal of the modules, so that a pole can typically be outfitted with a small assortment of modules and not changed casually. However, the cap 9 is removable for adding or deleting modules.

There are two types of modules. The first type ends in a hook, suitable for hanging a reservoir of medical fluids. In FIG. 1, a module 3 of the first type is in a position suitable for hanging a bag of intravenous solution. Another module 5 is identical but oriented in a different direction. Another module 4 is also of the first type, but it is mounted low on the pole where it would typically be used to support a drainage bag that receives body fluids.

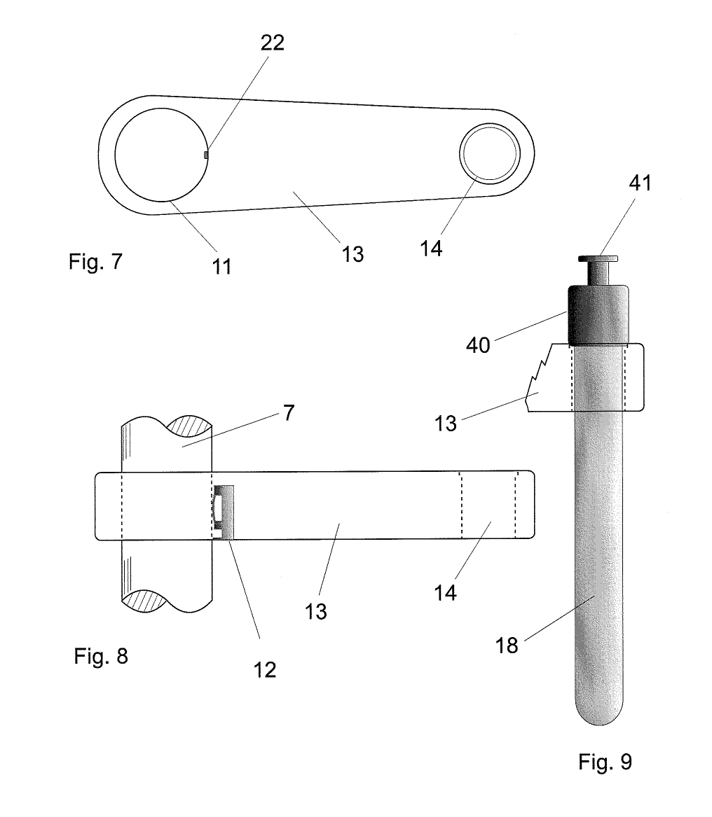

The second type of module has no hook, but contains a rod hole at the end of the module furthest from the pole. FIG. 7 shows a module that is of the second type, having a rod hole 14 but not a hook In FIG. 1, a module 1 of the second type has a rod that is part of the base of a lamp 19 inserted in the rod hole. Another example module 2 has a tray 20 held by a rod that is part of the tray in the rod hole. Yet another module 17 has a rod 18 inserted in the rod hole so it can be hooked to some other apparatus, such as a bed rail with the objective of preventing movement, or a gurney or wheelchair frame with the objective of trailering the pole behind the gurney or wheelchair.

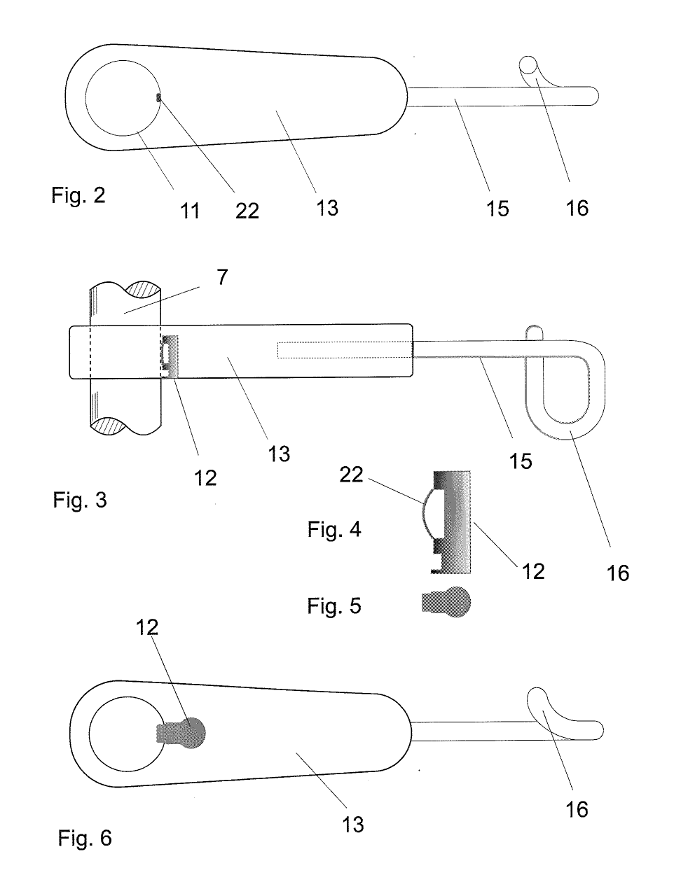

FIG. 2 shows a module fitted with a hook, so it is the first type. In all modules, there is a pole hole 11 in the body 13, through which the vertical pole 7 will pass. For convenient reference, the end of the module with the pole hole is called the “pole end” and the opposite end is called the “distal end”. In FIG. 2, a metal hook rod 15 has been inserted into the body 13 at the time when the body was molded, which hook rod 15 is bent to form a hook 16, making this module of the first type. It is a manufacturing convenience to make one module with the same general shape that can be used for both types, and then either mold a hook rod 15 into it as in FIG. 2, or mold a rod hole 14 into it as in FIG. 7. In another embodiment, a combination module could be made with both a rod hole parallel to the pole hole, and a hole orthogonal to the pole hole to receive the hook rod. If such a combination module body is manufactured, those which are turned into the first type will end up with the rod hole 14 blocked by the hook rod 15 so they cannot be used as the second type.

FIG. 3 shows a side view of module of the first type. A metal hook rod 15 has been molded into the body 13 at the distal end. The module is mounted on a pole 7. There is a springy insert 12 in the wall of the pole hole 11, pressing resiliently against the pole 7. The body 13 of the module is formed by injection molding of rigid polymer, of which several suitable types are known in the art. The substance will simply be referred to as “plastic”. The plastic may have colour added, and a range of colours may be useful to users for the purpose of distinguishing which department of a hospital lays claim to the module. The plastic may also have antimicrobial material added, for a sanitary benefit.

FIG. 4 shows details of the springy insert 12. It is typically molded of the same plastic material as the body 13, and typically can be molded at the same time by making it as an attachment by a filament to the body 13. It has a bowed section 22 that will press against, and be compressed by, the pole 7. The bowed shape provides springiness, because the plastic is resilient and attempts to retain the shape into which it was molded, but can bend under pressure and the void behind the bowed section 22 allows the bowed section 22 to deform while retaining its inclination to return to its original shape. In manufacturing, when the molded product emerges from the mold, the springy insert 12 can be detached from the filament (not shown) and pressed into a matching recess in the body 13. In use, the springy insert 12 presses against the pole 7 with enough force so that the friction between the pole 7 and both the springy insert 12 and the rim of the pole hole 11 is sufficient to prevent sliding of the module under the force of gravity alone, but not so strongly that a hand cannot slide the module along the pole.

FIG. 5 shows that the springy insert 12 has a keyhole shape. FIG. 6 shows that the body 13 has a corresponding keyhole-shaped recess molded into it from one side, into which the springy insert 12 is pressed. The recess does not pass all the way through the body 13. Typically, in use the opening of the recess for the springy insert 12 will be on the bottom side of the body 13, so that downward motion of the module under gravity or while being adjusted will not cause the springy insert 12 to slide upwards and out of the recess. The springy insert 12 cannot move into the pole hole 11 because it is held by the keyhole shape, and it cannot move upwards because the recess is a blind channel. Optionally, to prevent the springy insert 12 from moving downwards, it can be further held in place by means known in the art of plastics, such as electrosonic welding, or gluing.

The pole hole 11 is slightly larger than the diameter of the pole 7, so that the module fits loosely on the pole 7. In other words, since the pole is vertical the module can rotate a few degrees in the vertical plane. A rotation angle in the range of 2 to 6 degrees has been found to be effective, but the invention is not limited to that range. With no weight on the module, the module would tend to slide down the pole if it were not for the springy insert 12. However, the springy insert 12 prevents that situation. In operation with the weight of paraphernalia on the hook 16 or other attachment that extends from the body 13 of the module, the module rotates a few degrees so the distal end moves downward and the module is then not orthogonal to the pole. After such rotation, the body 13 is not horizontal, and the force of gravity acting on the body 13 includes a vector component that is horizontal and is pressing into the pole. The horizontal pressure against the pole 7 by the rim of the pole hole 11 causes the module to grip the pole 7. The bottom rim presses into the pole 7 on the side of the body 13 that holds the paraphernalia, and the upper rim presses into the pole 7 on the other side. As the weight of the paraphernalia increases, the grip against the pole 7 increases. Conversely, as the weight of the paraphernalia decreases, as when a supported reservoir of intravenous solution becomes empty, the grip against the pole 7 decreases, but so does the gravitational pull downwards. With an empty reservoir, the weight of the reservoir and the tubing running from it to the patient is generally sufficient to hold the module in place, and the placement of the module is further maintained by the pressure of the springy insert 12 against the pole 7.

The module can be positioned vertically anywhere on the pole 7, and may be rotated about the axis of the pole 7 so its horizontal axis is in any direction, simply by gripping it and moving it. Unlike the prior art, there is no thumbscrew to deal with, so positioning of the present invention is a one-hand operation.

Typical dimensions of a module have a body 13 that is 5 inches long and 1.5 inches wide at the pole end, typically but not necessarily tapering slightly towards the distal end. A suitable thickness of the body 13 is 0.75 inch, and it also may taper slightly towards the distal end. A common diameter of solid poles in current use is 1 inch. Some telescoping poles have that dimension in their upper segment. By using that common size the support brackets can be retrofitted to existing poles, although they will not be able to pass the telescoping joint where the pole diameter changes. The pole hole 11 can be made in a variety of sizes to fit various poles, especially for retrofitting existing poles.

The hook rod 15 that forms the hook typically projects 2.5 inches from the distal end of the body 13 before its first bend, and then curves around so that the point of support of paraphernalia at the bottom of the hook is about 2 inches from the end of the body 13, which is about 6 inches from the axis of the pole 7. A desirable style of hook 16, as is well known in the art, has the end of the hook slightly above the horizontal hook rod 15 and spaced away from the horizontal hook rod 15 by about 0.25 inch, so that the paraphernalia must be lifted with a turn to remove it from the hook, and therefore the paraphernalia is unlikely to be unhooked accidentally. The hook rod 15 can be inserted into the body 13 at the time of molding, and preferably the hook rod 15 bears means to hold it firmly in the body 13, such as a notch or circumferential groove, or splines, or knurling, or an enlarged head.

The rod hole 14 is adapted to receive various paraphernalia, and would typically be made 0.5 inch diameter, which has been found to suit several existing paraphernalia that would be desirable on the pole. Some paraphernalia, such as trays with a rod on the bottom, are not currently produced and would be a new product to complement this invention, and existing products could have a rod welded or otherwise fastened to them. FIG. 8 shows a side view of the module with pole 7 passing through body 13, and a rod 18 inserted in rod hole 14.

When the rod hole 14 is used with a straight rod 18, for hooking onto a bed, gurney, wheelchair or other hospital equipment, the rod may be held in place by pressure fit, or could be permanently inserted with splines on the surface of the rod within the rod hole. Most conveniently and flexibly, the rod would have a head, resembling a large nail, that will not pass through the rod hole and holds the rod in place by resting on the upper surface of the body 13. FIG. 9 shows a rod 18 typically made of metal, and a head made of the same plastic material as the module body 13. The head 40 can advantageously include a nail-shaped post 41 as part of the molded shape, for hanging paraphernalia or tags.

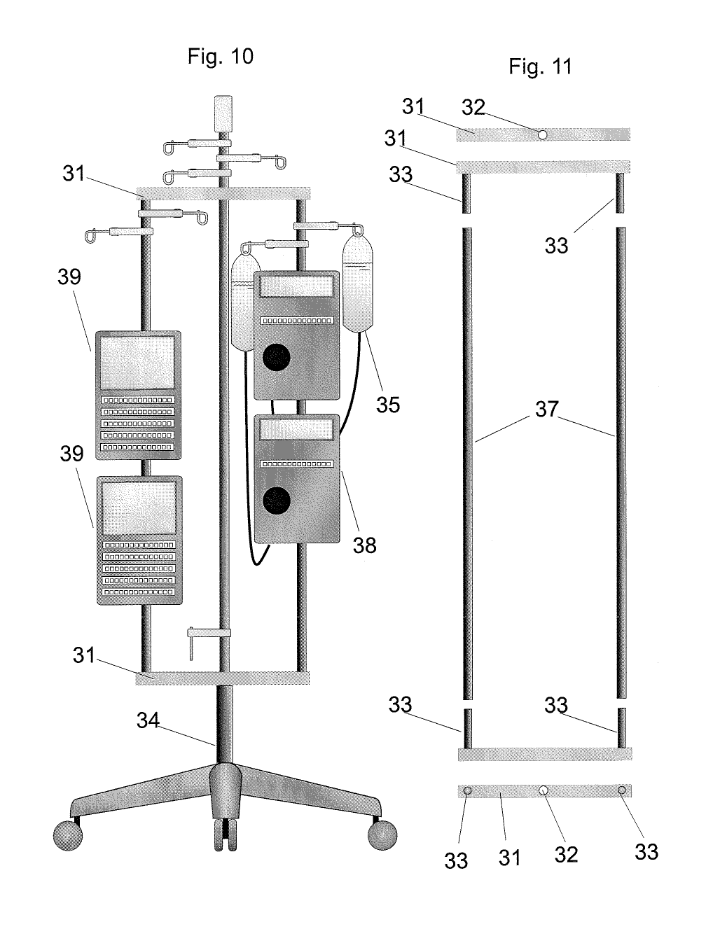

Although the modules can be spaced closely on the pole, and in different directions, the paraphernalia require space and that puts a limit on how much can be held by one pole. In some cases, particularly in operating theatres, there is a need for the pole to support pumps and monitors that have their own means for attachment to a pole and do not require a module. To expand the capacity of a pole rather than bring in another pole with its wheeled platform taking floor space, the capacity of a pole can be approximately tripled by the apparatus shown in FIG. 10 and FIG. 11. A pair of essentially identical cross-members 31, have a hole 32 through which the pole 7 will fit so that the pole 7 will slide through hole 32 but is not a loose fit. Preferably the clearance between the pole 7 and the hole 32 is in the range 0.002 to 0.005 inch. The cross-members 31 near each end have rods 33 fastened tightly to them by any means, including welding, friction fit, threaded rod in threaded hole, riveting, set-screw, glue, billet machining or unitary casting. The diameter of the rods 33 matches the inside diameter of the tubing that forms the sub-poles 37. The sub-poles 37 have the outside diameter suited to fit the modules in this invention, and that diameter most conveniently is the same diameter as the pole 7 so the same modules fit both sub-poles and poles. The sub-poles 37 may be the same material as the pole 7. Each rod 33 should fit into sub-pole 37 tightly enough to avoid rattling or wiggling of the sub-pole 37 on the rod 33, but loosely enough that the rods 33 with attached cross-member 31 can be pulled out to allow modules to be added or taken away from the sub-pole 37 by sliding modules over an end of the sub-pole.

In another embodiment of the apparatus with the two sub-poles 37, the cross-members 31 do not have rods 33 attached but instead have blind holes where the rods 33 are illustrated in FIG. 11. In this embodiment, the sub-poles 37 fit snugly into the blind holes so that disassembly to add modules is not impossible, but looseness in the blind holes would be undesirable. The cross-member for this embodiment should be thick enough to allow deep enough blind holes to solidly contain the sub-poles 37. Since the typical sub-pole 37 is one inch in diameter, a one-and-one-half inch square solid bar has been found suitable for the cross-member 31, but it could alternatively be rectangular and two or more inches in height. To keep the total weight low, aluminum is a desirable material for the cross-member.

FIG. 10 illustrates how paraphernalia, such as pumps 38 and monitors 39, can be added to the same pole using the attachment means that are incorporated into such equipment, and the pole 7 and sub-poles 37 can also hold the reservoirs 35 of medical fluids using the modules of this invention.

In FIG. 10, the apparatus comprising two sub-poles 37 is held in place in the vertical direction by the lower cross-member 31 resting on a sleeve 34 that fits over the pole 7. The sleeve 34 may be part of an oxygen cage holder, an apparatus commonly attached to wheeled poles in hospitals. In another embodiment, the lower cross-member 31 rests directly on the wheeled platform.

The invention and its advantages will be apparent from the foregoing description. It will be apparent that various changes may be made in the form, construction and arrangement of parts of the invention without departing from the spirit and scope thereof, which is defined by the appended claims. Although the intent of this invention is primarily for use in a medical setting, the simplicity and usefulness of its function has the potential for use in other applications.

What is claimed is:

1. A support bracket for supporting paraphernalia on a pole, comprising:
an elongated body having, near one end of said body, a hole through which a pole may slide, the axis of said hole being perpendicular to the direction of elongation of said body;
support means for paraphernalia extending from said body in the direction of elongation of said body, from the end of said body farthest from the end containing said hole;
a resilient member protruding into said hole far enough to resiliently press against said pole;
wherein the rim of said hole frictionally engages said pole with the force of the horizontal vector in said body of the gravitational force on said paraphernalia and said body when said body is not horizontal.

2. A support bracket as recited in claim 1, in which said support means is a hook.

3. A support bracket as recited in claim 1, in which said body is made of plastic.

4. A support bracket as recited in claim 3, in which said plastic is impregnated with an antimicrobial compound.

5. A support bracket for supporting paraphernalia on a pole, comprising:
an elongated body having, near one end of said body, a first hole through which said pole may slide, the axis of said first hole being perpendicular to the direction of elongation of said body;
a second hole near the end of said body farthest from said first hole having its axis parallel to said first hole;
a resilient member protruding into said first hole far enough to resiliently press against said pole;
wherein the rim of said first hole frictionally engages said pole with the force of the horizontal vector in said body of the gravitational force on said paraphernalia and said body when said body is not horizontal.

6. A support bracket as recited in claim 5, in which a rod fits snugly into said second hole and extends in at least one direction perpendicular to the long axis of said body.

7. A support bracket as recited in claim 5, in which a rod that is part of any item of paraphernalia fits into said second hole to indirectly mount said item on said pole.

8. A support bracket as recited in claim 5, in which said body is made of plastic.

9. A support bracket as recited in claim 8 in which said plastic is impregnated with an antimicrobial compound.

10. An apparatus for supporting paraphernalia, comprising:
a pole mounted vertically on a frame comprising wheels;
at least one support bracket for supporting paraphernalia mounted on said pole by having said pole pass slidingly through a hole in the body of said support bracket, said hole being perpendicular to the longest dimension of said support bracket;
connection means for attaching said paraphernalia at the end of said body farthest from said pole;
a resilient member protruding into said hole far enough to resiliently press against said pole;
wherein the rim of said hole frictionally engages said pole when said body is not horizontal.

11. An apparatus as recited in claim 10, in which said connection means is a rod with a hooked end inserted in said body radially in relation to said pole.

12. An apparatus as recited in claim 10, in which said connection means is a hole in said body, said hole being adapted to receive a rod that is part of an item of paraphernalia.

13. An apparatus for holding a support bracket as claimed in claim 1 and for also holding paraphernalia having their own means of attachment to a pole, comprising:
an upper cross-member and a lower cross-member;
a pair of tubes joining said upper cross-member to said lower cross-member, upon which both said support brackets and said paraphernalia are mountable;
wherein a hole in each cross-member slidingly accepts a pole.

14. An apparatus for holding a support bracket as claimed in claim 5 and for also holding paraphernalia having their own means of attachment to a pole, comprising:
an upper cross-member and a lower cross-member;
a pair of tubes joining said upper cross-member to said lower cross-member, upon which both said support brackets and said paraphernalia are mountable;
wherein a hole in each cross-member slidingly accepts a pole.
